(19) United States
(12) Patent Application Publication
(10) Pub. No.: US 2016/0183852 A1
(43) Pub. Date: Jun. 30, 2016

(54) SYSTEM AND METHOD FOR DETERMINING A STATE OF A BODY

(71) Applicant: Priya E. JOSEPH, San Carlos, CA (US)

(72) Inventor: Priya E. JOSEPH, San Carlos, CA (US)

(21) Appl. No.: 14/981,717

(22) Filed: Dec. 28, 2015

Related U.S. Application Data
(60) Provisional application No. 62/096,986, filed on Dec. 26, 2014.

(51) Int. Cl. A61B 5/1455; A61B 5/00; A61B 5/145

(52) U.S. Cl. CPC A61B 5/1455; A61B 5/14532; A61B 5/0013; A61B 5/6831; A61B 5/6832; A61B 5/6898; A61B 2562/0238; A61B 2576/00

(57) ABSTRACT

The invention provides a system for determining a state of a body. A location unit that includes a carrier component having a front side and an opposing back side, an attachment device having a first portion connected to the carrier component and a second portion securing the carrier component to the body with the back side facing a surface of the body. The location unit has a camera secured to the carrier component on the back side thereof for capturing video images of a feature of the body at or below the surface of the body. A server computer system has a recording system that stores the video images on a recording medium and an analysis system connected to the recording medium and being configured to analyze the image to determine the state of the body and report back to the location device.

### CROSS-REFERENCE TO RELATED APPLICATIONS

This application claims priority from U.S. Provisional Patent Application No. 62/096,986, filed on Dec. 26, 2014, all of which is incorporated herein by reference in its entirety.

### BACKGROUND OF THE INVENTION

1). Field of the Invention

This invention relates to system and method for determining a state of a body.

2). Discussion of Related Art

It is known that people who are diabetic have a need for controlling their blood sugar levels through diet and insulin supplementation. In order to determine whether their blood sugar levels are too high or too low, it is often required to make a pin prick on their finger in order to remove some blood from their body for analysis. Such a pin prick may be somewhat painful and a person may not want to repeat the process on regular basis. As a result, it may not be possible for the person to monitor their blood sugar levels in real time.

Blood sugar levels of diabetics are an example of a state of a body that requires monitoring. Another example of a state of a body that may require monitoring may for example be sugar levels of fruit to determine their ripeness, or the inclusion of pesticides in fruits and vegetables.

U.S. patent application Ser. No. 14/350,798 describes an automated personal medical diagnostic system and arrangement, including: at least one sensor configured to measure and/or sense at least one physiological condition and generate or acquire sensor data; at least one computing device configured to process at least a portion of the sensor data and generate diagnostic data based at least partially on the sensor data; and at least one user interface configured for user interaction; wherein the diagnostic data at least partially comprises at least one of the following: indicator data, medical diagnostic data, trigger data, or any combination thereof. A method for automated medical diagnosis is also disclosed.

### SUMMARY OF THE INVENTION

The invention provides a system for determining a state of a body including a location unit that includes a carrier component having a front side and an opposing back side, an attachment device having a first portion connected to the carrier component and a second portion securing the carrier component to the body with the back side facing a surface of the body and a camera secured to the carrier component on the back side thereof for capturing at least an image of a feature of the body at or below the surface of the body, a recording medium, a recording system storing the image on the recording medium and an analysis system connected to the recording medium and being configured to analyze the image to determine the state of the body.

The system may further include that the attachment device is a strap that is securable around the body.

The system may further include that the second portion if the attachment device is an adhesive that attaches to the body.

The system may further include that the feature that is captured is below the surface of the body.

The system may further include that the feature at least includes a sugar level.

The system may further include that the camera captures a video, the recording medium stores the video, and the analysis system analyzes the video to determine the state of the body.

The system may further include that the analysis system performs a spectrographic analysis of the video.

The system may further include that the image is a baseline image and the state of the body is a baseline state of the body, the camera captures at least a follow-up image of the feature of the body at or below the surface of the body with the camera secured to the carrier component on the back side thereof, the recording system stores the follow-up image on the recording medium, the analysis system analyzes the follow-up image to determine a follow-up state of the body, and compares the follow-up state of the body with the baseline state of the body to determine a change of state of the body.

The system may further include that the recording medium forms part of the location unit. The location unit may further include a network interface device connected to the recording device and a data forwarding system that forward the image over the network interface device, further including a server computer system that includes a processor, a network interface device connected to the processor, a computer readable medium connected to the processor and a set of instructions on the computer readable medium that are executable by the processor. The set of instructions may include a data reception system that receives the image from the network interface device of the location unit over the network interface device of the server, wherein the analysis system forms part of the set of instructions of the server.

The system may further include that the data forwarding system delays the transmission of the image.

The system may further include that the location unit further includes an output to inform a user of the state of the body.

The invention may also provide a method of determining a state of a body including securing a carrier component of a location unit to a body using an attachment device of the location unit having a first portion connected to the carrier component and a second portion connecting the carrier component to the body, the carrier component having a front side and an opposing back side the back side facing a surface of the body, capturing at least an image of a feature of the body at or below the surface of the body with a camera of the location unit secured to the carrier component on the back side of the carrier component, storing the image on a recording medium and analyzing the image to determine the state of the body using an analysis system connected to the recording medium.

The method may further include that the attachment device is a strap that is securable around the body.

The method may further include that the second portion if the attachment device is an adhesive that attaches to the body.

The method may further include that the feature that is captured is below the surface of the body.

The method may further include that the feature at least includes a sugar level.

The method may further include that the camera captures a video, the recording medium stores the video, and the analysis system analyzes the video to determine the state of the body.

The method may further include that the analysis system performs a spectrographic analysis of the video.

The method may further include that the image is a baseline image and the state of the body is a baseline state of the body, further including capturing at least a follow-up image of the feature of the body at or below the surface of the body with the camera secured to the carrier component on the back side thereof; storing the follow-up image on the recording medium, analyzing the follow-up image to determine a follow-up state of the body using the analysis system connected to the recording medium and comparing the follow-up state of the body with the baseline state of the body to determine a change of state of the body.

The method may further include that the recording medium forms part of the location unit and the location unit, further including forwarding the image over the network interface device and receiving the image from the network interface device of the location unit over a network interface device of a server, wherein the analysis system forms part of a set of instructions of the server.

The method may further include delaying the transmission of the image.

The method may further include providing an output to the location unit to inform a user of the state of the body.

### BRIEF DESCRIPTION OF THE DRAWINGS

The invention is further described by way of example with reference to the accompanying drawings, wherein:

FIG. 1 diagrammatic representation of a system, according to an embodiment of the invention, for determining a state of a body;

FIG. 2 is a block diagram of a location unit forming part of the system;

FIG. 3 is a front view of the location unit illustrating a front surface and display thereof;

FIG. 4 is a flow chart illustrating how baseline data is collected using the system;

FIG. 5 is s flow chart illustrating how follow-up data is collected using the system;

FIG. 6 is flow chart illustrating a process that is carried out after the follow-up data is collected and the baseline and follow-up data is then analyzed and reported;

FIG. 7 are charge-coupled device (CCD) images that are captured and spectrographically analyzed;

FIG. 8 is a block diagram of a server computer system that is a machine forming part of the system;

FIG. 9 is a side view of a system according to another embodiment of the invention; and

FIG. 10 is a side view of a system according to further embodiment of the invention.

### DETAILED DESCRIPTION OF THE INVENTION

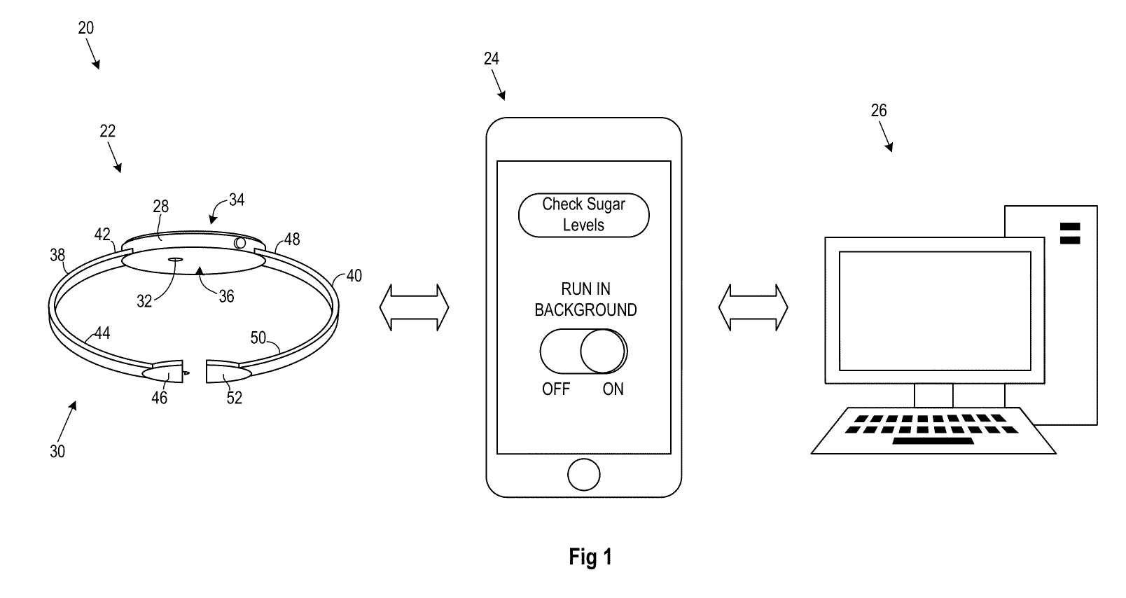

FIG. 1 of the accompanying drawings illustrates a system 20, according to an embodiment of the invention, for determining a state of a body, including a location unit 22, a mobile phone 24, and a server computer system 26.

The location unit 22 includes a carrier component 28, an attachment device 30, and a camera 32.

The carrier component 28 has a front side 34 and an opposing back side 36.

The attachment device 30 includes first and second straps 38 and 40. The strap 38 has a first portion 42 secured to the carrier component 28, a second portion 44 extending from the first portion 42, and a first clasp 46. The second strap 40 has a first portion 48 secured to the carrier component 28 on a side thereof opposing the first portion 42 of the first strap 38, a second portion 50 extending from the first portion 48, and a second clasp 52.

The camera 32 is secured to the carrier component 28 on the back side 36 thereof. The camera 32 is positioned to capture images, including video images, from the back side 36 of the carrier component 28.

In use, a user positions the back side 36 of the carrier component 28 on their wrist. The user then brings the first and second straps 38 and 40 around their wrist and secures the first and second clasps 46 and 52 to one another. The second portions 44 and 50 of the first and second straps 38 and 40 thereby secure the carrier component 28 to the user's wrist. The camera 32 is then positioned to capture a video of a feature of a body of the person at or below a surface of their skin facing the back side 36 of the carrier component 28. The feature that is capture may for example represent blood sugar levels within veins and arteries below the surface of the skin.

The mobile phone 24 is communicatively coupled to the location unit 22 by way of a wireless link. Data can exchange bidirectionally between the location unit 22 and the mobile phone 24.

The server computer system 26 is connected over the Internet to the mobile phone 24. The server computer system 26 is used for high end data analysis, for video analysis by a physician and for data reporting back to the user. The server computer system 26 and the mobile phone 24 can communicate data bidirectionally between them.

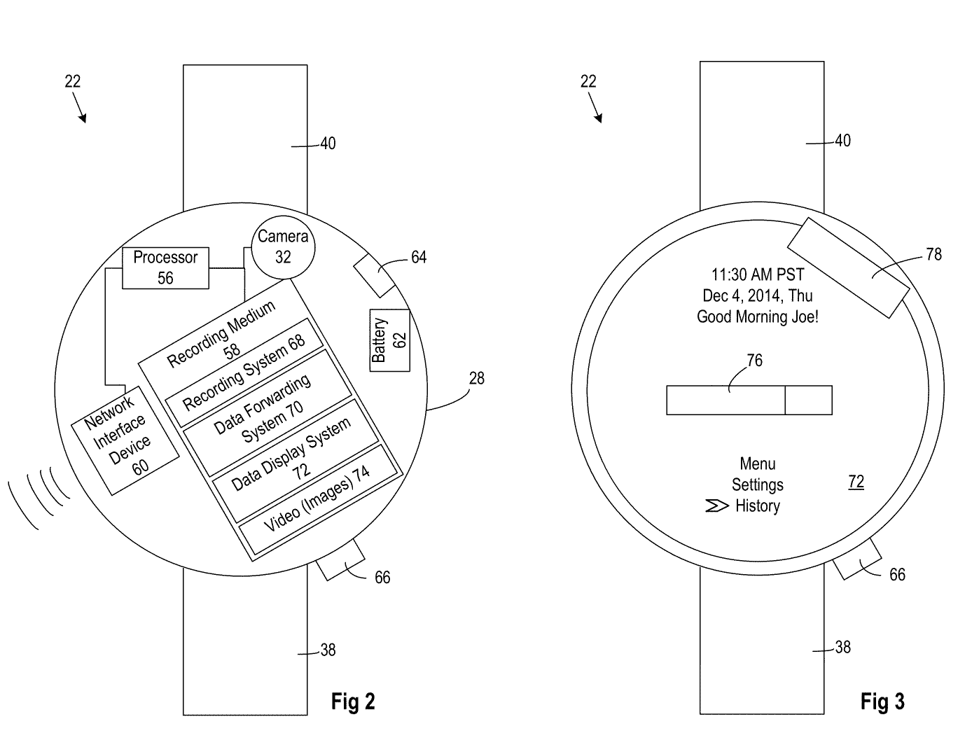

As shown in FIG. 2, the location unit 22 further includes a processor 56, a recording medium 58, a network interface device 60, a battery 62, a connector 64 for purposes of providing power and charging the battery 62, and one or more human interface elements such as a button 66. The camera 32, recording medium 58 and network interface device 60 are connected to the processor 56. The recording medium 58 has a set of instructions stored thereon including a recording system 68, a data forwarding system 70, a data display system 72 and video data 74. The recording system 68, data forwarding system 70 and data display system 72 are executable by the processor 56. The recording system 68 stores videos captured by the camera 32 within the video data 74. The data forwarding system 70 transmits the video data 74 over the network interface device 60 to the mobile phone 2 shown in FIG. 1. The data display system 72 receives data from the mobile phone 24 and displays the data to the user. The battery 62 is connected to the connector 64 so that it can be recharged when the connector 64 is connected to a power supply. The battery 62 provides power to the camera 32, processor 56 and network interface device 60.

A shown in FIG. 3, the location unit 22 includes a display 72 on the front side 34 of the carrier component 28. The user may use the button 66 to scroll through a menu. The user's sugar levels are displayed using a graphic 76 on the home page. The user can use the button 66 to access other pages for adjusting settings and obtain an history of their blood sugar levels.

The location unit 22 further includes a solar module 78 on the front side 34 of the carrier component 28. The solar module 78 converts light energy to a voltage and charges the battery 62 shown in FIG. 2.

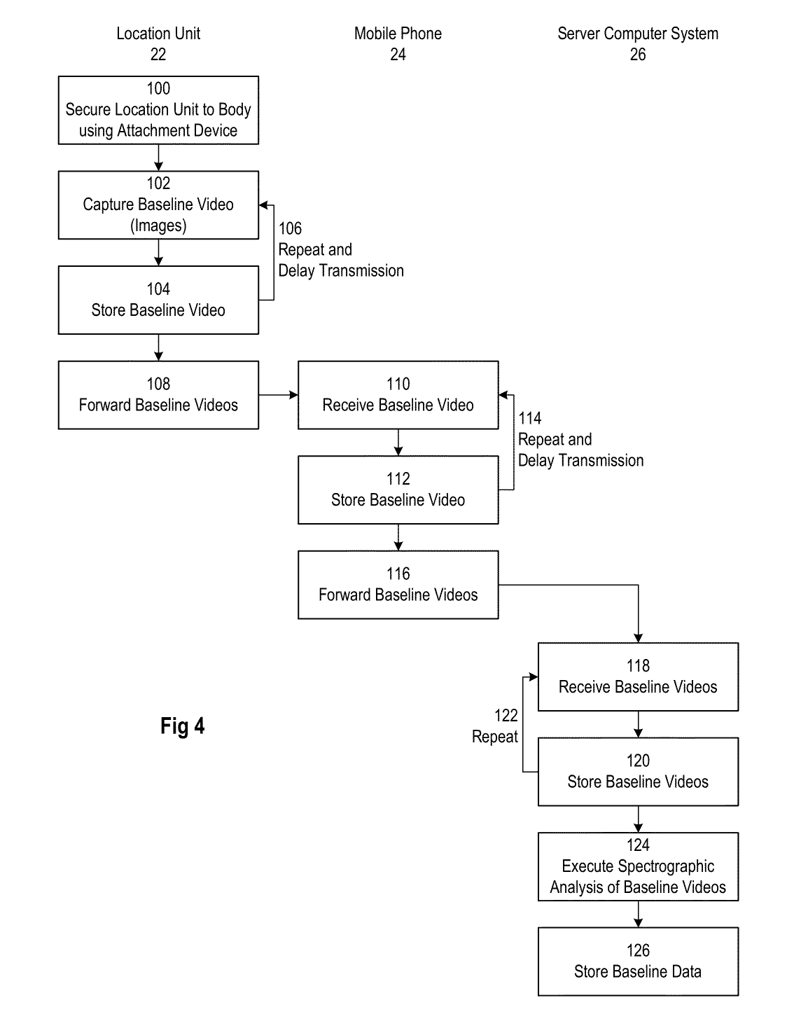

FIG. 4 shows how baseline data is established using the system 20 of FIG. 1. At 100, the user secures the location unit 22 to their body using the attachment device as described with reference to FIG. 1. At 102, the camera 32 captures a baseline video (images). At 104, the processor 56 stores the baseline video within the video data 74. Step 106 indicates that steps 102 and 104 are repeated while data transfer is delayed. Data transfer may for example be delayed for 15 minutes while video data is accumulated. At 108, the processor 56 forwards the baseline videos recorded within the video data 74 over the network interface device 60 to the mobile phone 24.

At 110, the mobile phone 24 receives the baseline videos from the location unit 22. At 112, the mobile phone 24 stores the baseline videos on a storage device thereof. Step 114 indicates that steps 110 and 112 are repeated while data transmission is delayed. Data transmission may for example be delayed for 24 hours while more baseline videos are collected and stored on the mobile phone 24. At 116, the mobile phone 24 forwards the baseline videos to the server computer system 26.

At 118, the server computer system 26 receives the baseline videos. At 120, the sever computer system 26 stores the baseline videos on a storage device. Step 122 indicates that steps 118 and 120 are repeated. Steps 118 and 120 may for example be repeated every 24 hours over a period of two weeks while more baseline videos are collected and stored by the server computer system 26.

At 124, the server computer system 26 executes a spectrographic analysis of baseline videos. A spectrographic analysis is preferably executed using the techniques described in Shih, Wei-Chuan, K. B. Bechtel, and Michael S. Feld. “Noninvasive glucose sensing with Raman spectroscopy,” Analytical chemistry of in vivo glucose measurements. Hoboken, N.J.: John Wiley & Sons (2009): 391-419 and Wadhwa, Neal, et al., “Phase-Based Video Motion Processing,” Phase-Based Video Motion Processing, SIGGRAPH 2013, which are incorporated herein by reference in their entirety. At 126, the server computer system 26 stores baseline data that is the result of the spectrographic analysis executed at step 124.

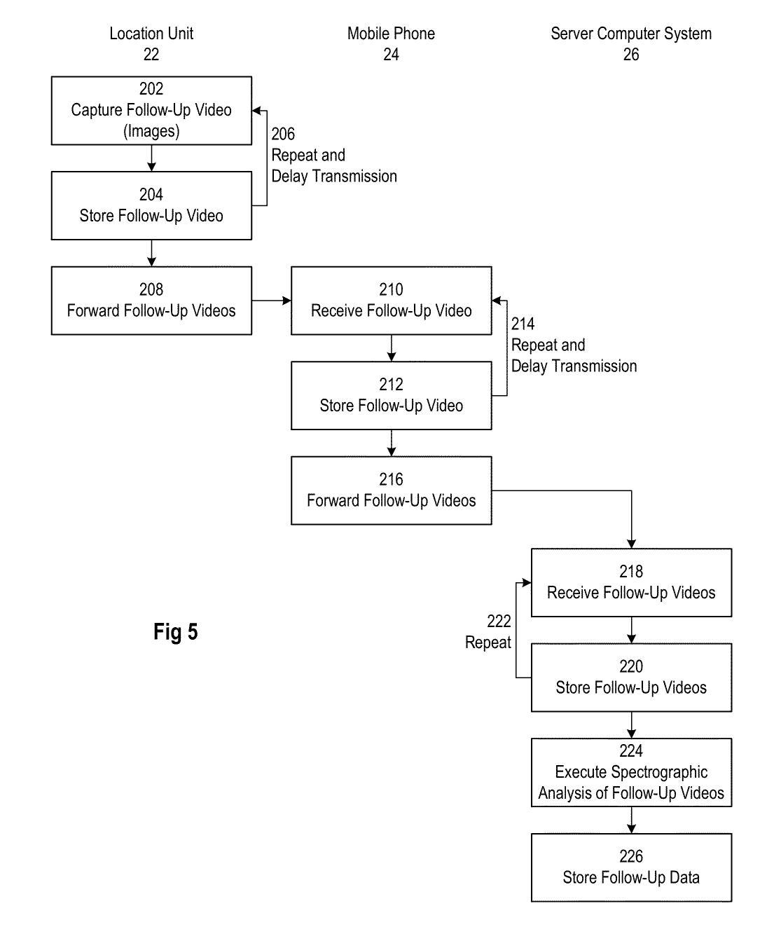

FIG. 5 illustrates how follow-up data is created after the baseline data is created in FIG. 4. At 202, the camera 32 captures a follow-up video (images). At 204, the processor 56 stores the follow-up video within the video data 74. Step 206 indicates that steps 202 and 204 are repeated while data transfer is delayed. Data transfer may for example be delayed for 15 minutes while video data is accumulated. At 208, the processor 56 forwards the follow-up videos recorded with-in the video data 74 over the network interface device 60 to the mobile phone 24.

At 210, the mobile phone 24 receives the follow-up videos from the location unit 22. At 212, the mobile phone 24 stores the follow-up videos on a storage device thereof. Step 214 indicates that steps 210 and 212 are repeated while data transmission is delayed. Data transmission may for example be delayed for one or two hours, or as needed, while more follow-up videos are collected and stored on the mobile phone 24. At 216, the mobile phone 24 forwards the follow-up videos to the server computer system 26.

At 218, the server computer system 26 receives the follow-up videos. At 220, the sever computer system 26 stores the follow-up videos on a storage device. Step 222 indicates that steps 218 and 220 may be repeated. More follow-up videos may be recorded as needed. Follow-up videos are typically analyzed within a shorter period than baseline videos in order to provide the user with faster feedback. Follow-up videos may for example be analyzed within 24 to 36 hours after they have been recorded.

At 224, the server computer system 26 executes a spectrographic analysis of follow-up videos. At 226, the server computer system 26 stores follow-up data that is the result of the spectrographic analysis executed at step 224.

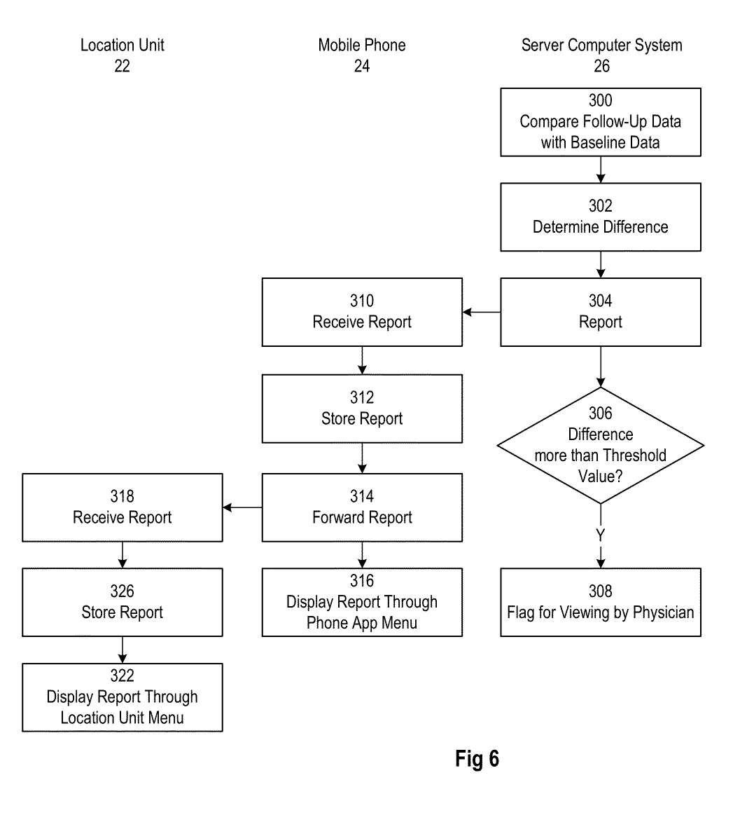

FIG. 6 illustrates the process that is carried out after the follow-up data and baseline data are collected. At 300, the server computer system 26 compares the follow-up data with the baseline data. At 302, the server computer system 26 determines the difference between the follow-up data and the baseline data. At 304, the server computer system 26 reports the difference to the mobile phone 24. At 306, the server computer system 26 determines whether the difference is more than a threshold value. If the difference is less than a threshold value, then the data is not flagged for viewing by a physician. If the difference is more than the threshold value, then the data is flagged for viewing at a physician at 308.

At 310, the mobile phone 24 receives the report from the server computer system 26. At 312, the mobile phone 24 stores the report within a storage medium. At 314, the mobile phone 24 forwards the report to the location unit 22. At 316, the user has the option to display the report through the app menu of the mobile phone 24.

At 318, the location unit 22 receives the report from the mobile phone 24. At 326, the location unit 22 stores the report within the recording medium. The report transmission at 304 and 314 and the storing of the report at 326 may all be accomplished while the mobile phone 24 and the location unit 22 are in “sleep” mode and therefore without any user interaction. At 322, the report is displayed through the location unit menu as described with reference to FIG. 3.

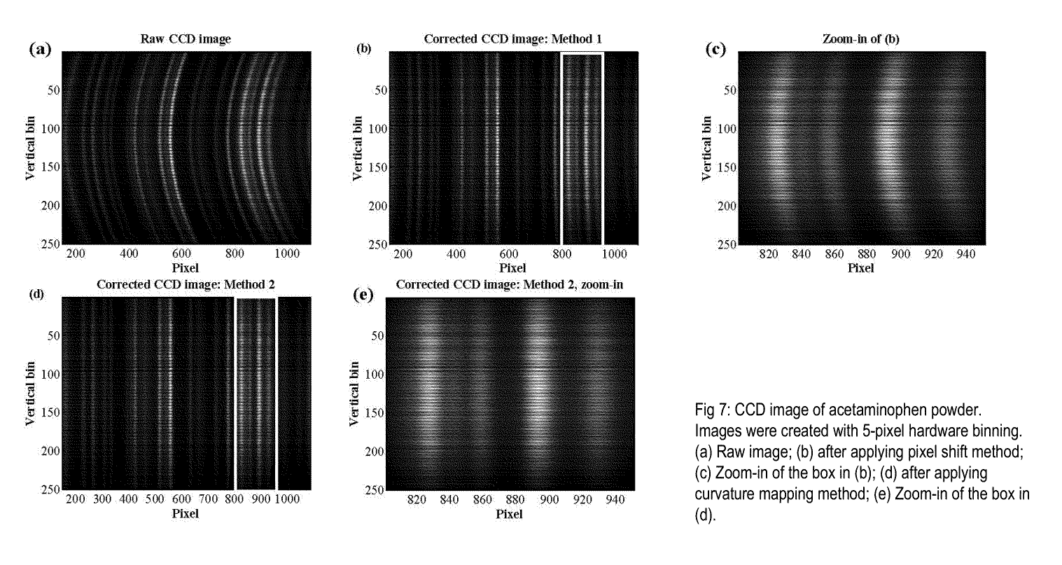

FIG. 7 shows examples of spectrographic images that are created at 124 and 224 in FIGS. 4 and 5. The spectrographic images include data that can be compared as described with reference to FIG. 6. The spectrographic images need not be displayed on a display of the server computer system 26 in order to analyze and compare baseline and follow-up data.

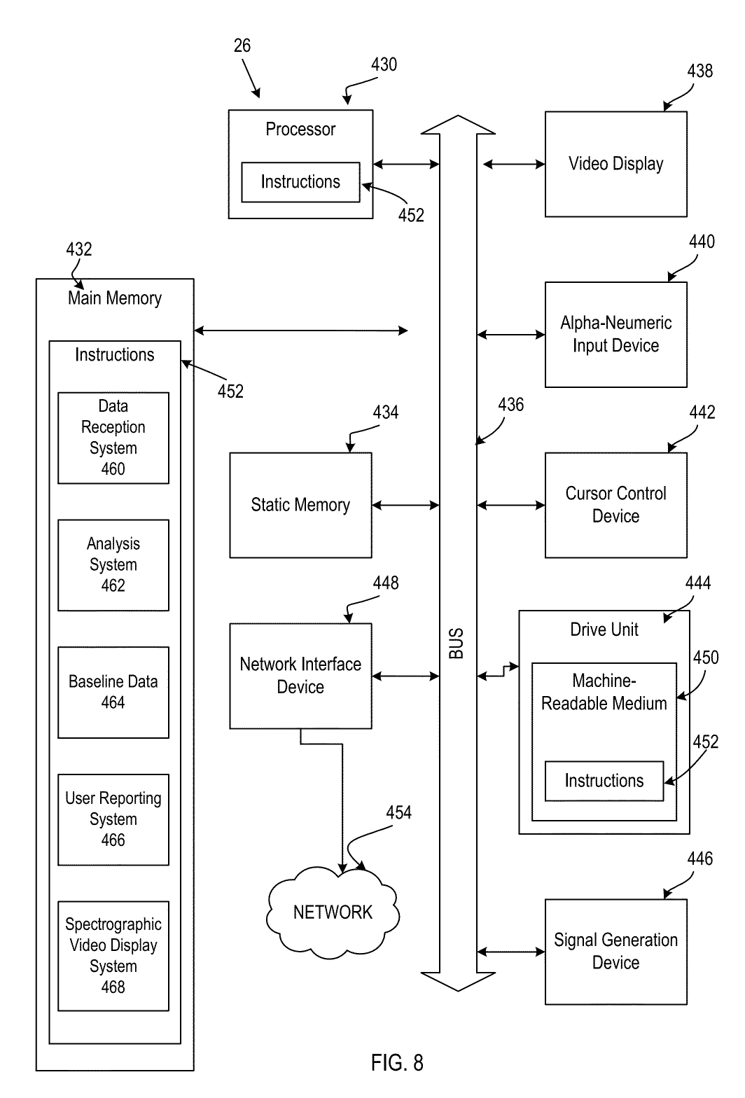

FIG. 8 shows a diagrammatic representation of a machine in the exemplary form of a server computer system 26 within which a set of instructions, for causing the machine to perform any one or more of the methodologies discussed herein, may be executed. In alternative embodiments, the machine operates as a standalone device or may be connected (e.g., networked) to other machines. In a network deployment, the machine may operate in the capacity of a server or a client machine in a server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The exemplary server computer system 26 includes a processor 430 (e.g., a central processing unit (CPU), a graphics processing unit (GPU), or both), a main memory 432 (e.g., read-only memory (ROM), flash memory, dynamic random access memory (DRAM) such as synchronous DRAM (SDRAM) or Rambus DRAM (RDRAM), etc.), and a static memory 434 (e.g., flash memory, static random access memory (SRAM, etc.), which communicate with each other via a bus 436.

The server computer system 26 may further include a video display 438 (e.g., a liquid crystal displays (LCD) or a cathode ray tube (CRT)). The server computer system 26 also includes an alpha-numeric input device 440 (e.g., a keyboard), a cursor control device 442 (e.g., a mouse), a disk drive unit 444, a signal generation device 446 (e.g., a speaker), and a network interface device 448.

The disk drive unit 444 includes a machine-readable medium 450 on which is stored one or more sets of instructions 452 (e.g., software) embodying any one or more of the methodologies or functions described herein. The software may also reside, completely or at least partially, within the main memory 432 and/or within the processor 430 during execution thereof by the server computer system 26, the memory 432 and the processor 430 also constituting machine readable media. The software may further be transmitted or received over a network 454 via the network interface device 448.

The instructions 452 include a data reception system 460, an analysis system 462, baseline data 464, a user reporting system 466 and a spectrographic video display system 468. The data reception unit 460 receives video data from the mobile phone 24. The analysis system 462 carries out spectrographic analysis on the video data 74. The baseline data 464 includes all data of baseline videos that have be spectrographically analyzed. The user reporting system 466 creates a report that is forwarded to the mobile phone 24. The spectrographic video display system 468 displays spectrographically analyzed video data 74 on the video display 438 for purposes of physician analysis. The analysis system 462 is also responsible for flagging data that require further analysis by a physician.

It can thus be seen that blood sugar levels can be monitored through a painless and non-invasive technique. The user can at least collect blood sugar data in real time. After baseline data is collected, the user may receive updates of their blood sugar levels on any interval. Moreover, the data that is reported to the user can be represented in a manner to inform the user of their blood sugar levels at any moment in time in the past.

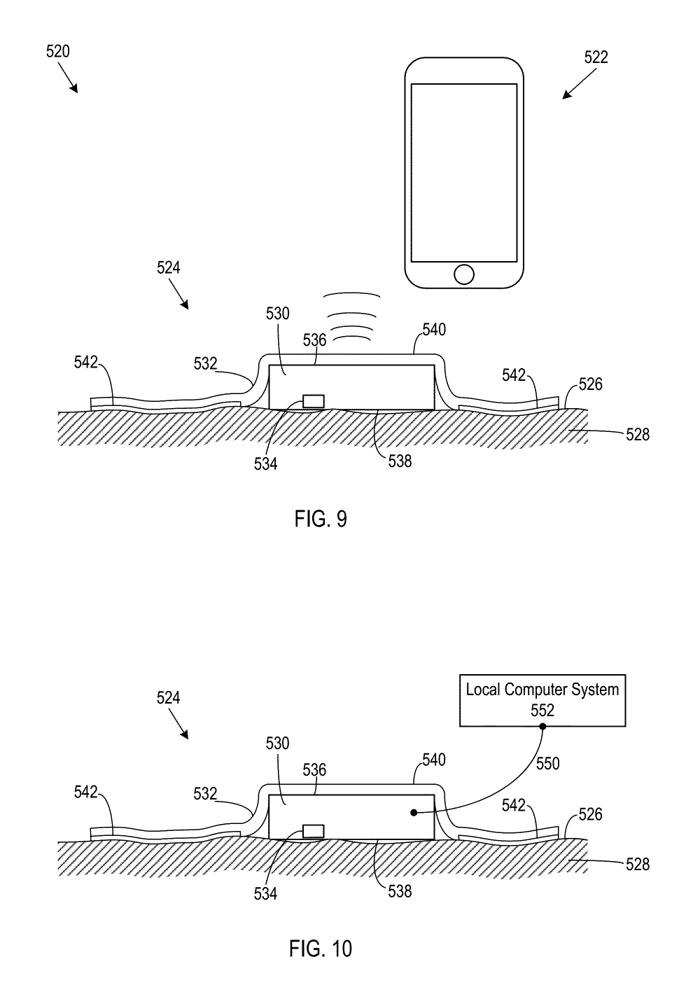

FIG. 9 illustrates an embodiment of a system 520 for determining a state of a body, according to another embodiment of the invention. The system 520 includes a mobile phone 522 and a location unit 524. The location unit 524 is secured to a surface 526 of a body 528. The system 520 in FIG. 9 is similar to the system 20 on FIG. 1 in that it is suitable for analyzing blood sugar levels of a person. The body 528 may for example be a part of a body of a person where blood sugar levels can be detected below a surface 526 of their skin. The system 520 may alternatively have a non-human application. For example, the system 520 may be used for determining a sugar level of a fruit in order to determine its ripeness. It may also be possible to use the system 520 for determining a feature of a body other than its sugar level. For fruits and vegetables, the system 520 may for example be used for determining pesticide levels.

The location unit 524 includes a carrier component 530, an attachment device 532 and a camera 534. The carrier component 530 has a front side 536 and back side 538. The attachment device 532 has a first portion 540 that is located over the front side 536 of the carrier component 530 and two adhesive second portions 542 distant from the carrier component 530. The camera 534 is located on the back side 538 of the carrier component 530.

In use, the carrier component 530 is located with the back side 538 on the surface 526 of the body 528. The adhesive second portions 542 are then attached to the surface 526 on opposing sides of the carrier component 530. The adhesive second portions 542 thereby secure the carrier component 530 to the body 528. The camera 534 captures images at or below the surface 526 of the body 528. A network interface device in the carrier component 530 may wirelessly communicate with the mobile phone 522 as described with reference to FIG. 1. The mobile phone 522 may in turn communicate with a sever computer system as hereinbefore described.

FIG. 10 describes a further embodiment that is similar to the embodiment of FIG. 9 and like reference numerals indicate like or similar components. The embodiment of FIG. 10 differs from FIG. 9 in that the location unit 524 is not wirelessly connected to a mobile phone, but instead is connected through a cable 550 to a local computer system 552. The embodiment of FIG. 10 may for example find application in a hospital, clinic or other setting where mobility of the user can be minimized.

While certain exemplary embodiments have been described and shown in the accompanying drawings, it is to be understood that such embodiments are merely illustrative and not restrictive of the current invention, and that this invention is not restricted to the specific constructions and arrangements shown and described since modifications may occur to those ordinarily skilled in the art.

What is claimed:

1. A system for determining a state of a body comprising:
a location unit that includes:
a carrier component having a front side and an opposing back side;
an attachment device having a first portion connected to the carrier component and a second portion securing the carrier component to the body with the back side facing a surface of the body; and
a camera secured to the carrier component on the back side thereof for capturing at least an image of a feature of the body at or below the surface of the body;
a recording medium;
a recording system storing the image on the recording medium; and
an analysis system connected to the recording medium and being configured to analyze the image to determine the state of the body.

2. The system of claim 1, wherein the attachment device is a strap that is securable around the body.

3. The system of claim 1, wherein the second portion if the attachment device is an adhesive that attaches to the body.

4. The system of claim 1, wherein the feature that is captured is below the surface of the body.

5. The system of claim 4, wherein the feature at least includes a sugar level.

6. The system of claim 1, wherein the camera captures a video, the recording medium stores the video, and the analysis system analyzes the video to determine the state of the body.

7. The system of claim 6, wherein the analysis system performs a spectrographic analysis of the video.

8. The system of claim 1, wherein the image is a baseline image and the state of the body is a baseline state of the body, the camera captures at least a follow-up image of the feature of the body at or below the surface of the body with the camera secured to the carrier component on the back side thereof, the recording system stores the follow-up image on the recording medium, the analysis system analyzes the follow-up image to determine a follow-up state of the body, and compares the follow-up state of the body with the baseline state of the body to determine a change of state of the body.

9. The system of claim 1, wherein the recording medium forms part of the location unit and the location unit further includes:
a network interface device connected to the recording device; and
a data forwarding system that forward the image over the network interface device, further comprising:
a server computer system that includes:
a processor;
a network interface device connected to the processor;
a computer readable medium connected to the processor; and
a set of instructions on the computer readable medium that are executable by the processor and include:
a data reception system that receives the image from the network interface device of the location unit over the network interface device of the server, wherein the analysis system forms part of the set of instructions of the server.

10. The system of claim 9, wherein the data forwarding system delays the transmission of the image.

11. The system of claim 1, wherein the location unit further includes:
an output to inform a user of the state of the body.

12. A method of determining a state of a body comprising:
securing a carrier component of a location unit to a body using an attachment device of the location unit having a first portion connected to the carrier component and a second portion connecting the carrier component to the body, the carrier component having a front side and an opposing back side the back side facing a surface of the body;
capturing at least an image of a feature of the body at or below the surface of the body with a camera of the location unit secured to the carrier component on the back side of the carrier component;
storing the image on a recording medium; and
analyzing the image to determine the state of the body using an analysis system connected to the recording medium.

13. The method of claim 12, wherein the attachment device is a strap that is securable around the body.

14. The method of claim 12, wherein the second portion if the attachment device is an adhesive that attaches to the body.

15. The method of claim 12, wherein the feature that is captured is below the surface of the body.

16. The method of claim 15, wherein the feature at least includes a sugar level.

17. The method of claim 12, wherein the camera captures a video, the recording medium stores the video, and the analysis system analyzes the video to determine the state of the body.

18. The method of claim 17, wherein the analysis system performs a spectrographic analysis of the video.

19. The method of claim 12, wherein the image is a baseline image and the state of the body is a baseline state of the body, further comprising:
capturing at least a follow-up image of the feature of the body at or below the surface of the body with the camera secured to the carrier component on the back side thereof;
storing the follow-up image on the recording medium;
analyzing the follow-up image to determine a follow-up state of the body using the analysis system connected to the recording medium; and
comparing the follow-up state of the body with the baseline state of the body to determine a change of state of the body.

20. The method of claim 12, wherein the recording medium forms part of the location unit and the location unit, further comprising:
forwarding the image over the network interface device;
receiving the image from the network interface device of the location unit over a network interface device of a server, wherein the analysis system forms part of a set of instructions of the server.

21. The method of claim 20, further comprising:
delaying the transmission of the image.

22. The method of claim 12, further comprising:
providing an output to the location unit to inform a user of the state of the body.
